(19) United States
(12) Patent Application Publication
(10) Pub. No.: US 2006/0161096 A1
(43) Pub. Date: Jul. 20, 2006

(54) Pessary applicator providing low placement

(76) Inventors: Thomas Ward Osborn III, Cincinnati, OH (US); Diana Lynne Gann, Lebanon, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/298,132

(22) Filed: Dec. 8, 2005

Related U.S. Application Data
(63) Continuation-in-part of application No. 10/860,910, filed on Jun. 4, 2004.

(51) Int. Cl. A61F 13/20

(52) U.S. Cl. 604/15

(57) ABSTRACT

A pessary applicator for positioning a pessary inside a vaginal cavity. The pessary applicator includes an outer member disposed co-axially with an inner member for insertion of the pessary into the vaginal cavity. The inner member is slidable within the outer member wherein at least 15% of a length of the pessary remains in the outer member when the inner member is fully engaged with the outer member to define a remaining portion of the pessary. The remaining portion of the pessary requires a force of less than about 0.30 N to be removed from the pessary applicator. Alternatively, when the inner member is fully engaged with the outer member to define a remaining portion of the pessary, the remaining portion of the pessary requires a force of less than about 0.04 N/mm to be removed from the outer member.

### CROSS REFERENCE TO RELATED APPLICATIONS

This application is a continuation-in-part of U.S. application Ser. No. 10/860,910, filed Jun. 4, 2004, pending.

### FIELD OF INVENTION

This invention relates to an applicator for proper placement of a urinary incontinent pessary into the vagina.

### BACKGROUND OF THE INVENTION

Urinary incontinence is a widespread problem among females. It is estimated that up to 50% of women occasionally leak urine involuntarily, and that approximately 25% of all women will seek medical advice at some point in order to deal with the problem. The two primary types of incontinence are stress incontinence and urge incontinence which have different origin and can occur as separate or combined conditions. Treating one condition does not necessarily mean the other condition has been treated. Incontinence is more prevalent among women who have given birth to one or more children. There are two primary demographic factors that are associated with female urinary incontinence: Child birth and aging.

Stress incontinence, the most common type of urinary incontinence, refers to the involuntary loss of urine resulting from abdominal pressure rise, occurring during exercise, coughing, sneezing, laughing, etc. When stress incontinence occurs, it is usually the result of the abnormal descent of the urethra and bladder neck below the level of the pelvic floor. While many different factors may contribute to the development of stress incontinence, stress incontinence is both aggravating and unpleasant for women, and it can also be embarrassing. Many women wear sanitary pads or diapers in order to deal with incontinence, though this is not a real solution to the problem. Wearing pad or diapers has are inconvenient and have side effects of skin irritation and odor limiting her social involvement and reducing her self esteem. Some cases of incontinence are treated surgically but have the normal risks associated with surgery, such as infection, and results are often not permanent.

One modality for non-surgical treatment used to reduce or prevent urinary incontinence in women includes devices which are located in the opening of the urethra and block the same. Shortcomings of these types of devices include the fact that they must be removed in order to empty the bladder and replaced with a new device which proves inconvenient and costly, they are susceptible to causing infection, and they often cause irritation to body tissue.

Another modality of non-surgical treatment involves the use of non-absorbent devices inserted into the vagina, either by a medical practitioner or by the woman herself. These devices are called pessaries. Pessaries are commonly used for the management of uterine prolapse and to provide vaginal support of the bladder or rectum. Many pessaries are designed to apply pressure against the bladder neck so as to inhibit or completely block the flow of urine through the urethra. A variety of such devices are known in the art for example, U.S. Pat. No. 5,618,256 to Reimer, entitled, “Device for Arrangement in the Vagina for Prevention of Involuntary Urination with Females and an Applicator for use in Insertion of the Device;” U.S. Pat. No. 5,785,640 to Kresch, entitled “Method for Treating Female Incontinence;” U.S. Pat. No. 4,920,986 to Biswas, entitled, “Urinary Incontinence Device;” U.S. Pat. No. 5,417,226 to I Juma, entitled, “Female Anti-Incontinence Device;” U.S. Pat. No. 5,386,836 to Biswas, entitled, “Urinary Incontinence Device;” and U.S. Pat. No. 5,007,894 to Enhorning, entitled, “Female Incontinence Device.” A number of devices are constructed so as to completely block the urethra and thus need to be removed or collapsed in order to allow the woman to urinate.

There are several important unaddressed needs associated with pessaries. These include reproducible and proper positioning of the pessary within the vagina by the consumer which is essential for the device to effectively and reliably relieve the symptoms associated with urinary incontinence. Proper placement is also important for comfort.

Recently there have been several pessary designs that have utilized conventional pessary type applicators to place pessaries within the vagina (WO2004/103213 and U.S. Pat. No. 6,090,099). This approach has the significant disadvantage of not reliably controlling the insertion of the pessary into the vagina where it comfortable and effective. Current pessary applicators place the pessary above the cervix where a pessary is not effective because it neither supports the prolapsed uterus nor does it apply pressure against the urethra nor does it help to straighten any “kinks” in the urethra. Furthermore, if the pessary is placed too low, it can become exceedingly uncomfortable. This approach is applicable to pessaries that are obtained by prescription and those purchased directly by the consumer. The approach is equally applicable to disposable and non-disposable pessary devices.

Tampon applicators adapted to pessaries have been used for inserting a pessary into a vaginal cavity. In the field of device applicators, “push” type pessary applicators are known. These applicators generally comprise, in a simple form, a pair of coaxial hollow cylinders or tubes. The larger outer tube serves as a vaginal insertion tube and houses a pessary. The smaller inner tube serves as a plunger which ejects the pessary from the outer tube.

It has been recognized that pessary applicators of the type described above have certain deficiencies which can be readily apparent to many users of such devices. In general, the position of the pessary within the vaginal cavity can directly impact the pessary's efficiency of reducing and/or eliminating urine leakage. For example, conventional “push” type applicators can place a pessary too high in the vaginal cavity forcing the device to the lateral side of the cervix. If the pessary is placed too high in the vagina and lodges to one side of the uterus, the reduction of urine leakage is not substantially improved because it cannot effectively support the urethra, uterus and bladder.

Alternatively, pessary applicators can place a pessary too low in the vaginal cavity. When the pessary is placed too low in the vaginal cavity, the pessary can cause bodily discomfort to the wearer because of the pressure exerted from the vaginal sphincter muscles.

To solve the above problem of positioning the pessary, it would be beneficial to provide a pessary applicator which properly places the pessary at a desired position in the vaginal cavity.

### SUMMARY OF THE INVENTION

The present invention encompasses a pessary applicator for positioning a pessary inside a vaginal cavity. The applicator includes an outer member disposed co-axially with an inner member for insertion of the pessary into the vaginal cavity. The inner member is slidable within the outer member wherein at least 15% of a length of the pessary remains in the outer member when the inner member is fully engaged with the outer member to define a remaining portion of the pessary. The remaining portion of the pessary requires a force of less than about 0.30 N to be removed from the outer member.

In another embodiment herein, a pessary applicator includes an outer member disposed co-axially with an inner member for insertion of the pessary into the vaginal cavity. The inner member is slidable within the outer member wherein at least 20% of a length of the pessary remains in the outer member when the inner member is fully engaged with the outer member to define a remaining portion of the pessary. The remaining portion of the pessary requires a force of less than about 0.50 N to be removed from the outer member.

In yet another embodiment, a pessary applicator includes an outer member disposed co-axially with an inner member for insertion of the pessary into the vaginal cavity. The inner member is slidable within the outer member wherein a length of at least 8 mm of the pessary remains in the outer member when the inner member is fully engaged with the outer member to define a remaining portion of the pessary. The remaining portion of the pessary requires a force of less than about 0.35 N to be removed from the outer member.

In another aspect, a pessary applicator includes an outer member disposed co-axially with an inner member for insertion of the pessary into the vaginal cavity. The inner member being slidable within the outer member wherein a portion of the pessary remains in the outer member when the inner member is fully engaged with the outer member to define a remaining portion of the pessary. The remaining portion of the pessary requires a force of less than about 0.04 N/mm to be removed from the outer member.

### BRIEF DESCRIPTION OF THE DRAWINGS

FIG. 1 is a perspective view along a longitudinal axis of a pessary applicator of the present invention.

FIG. 2 is a perspective view of the pessary applicator of the present invention.

FIG. 3 is a perspective view of the pessary applicator of the present invention when the pessary applicator is fully engaged.

FIG. 4 is a cross-sectional view along a longitudinal axis of the pessary applicator of the present invention.

FIG. 5 is a cross-sectional view along a longitudinal axis of the pessary applicator of the present invention.

FIG. 6 is a perspective view of an alternative embodiment of the pessary applicator of the present invention.

FIG. 7 is a cross-sectional view of an alternative embodiment of the pessary applicator of the present invention upon full engagement.

FIG. 8a is a perspective view of the Force Test Method equipment stand.

FIG. 8b is a perspective view of the Force Test Method equipment.

FIG. 8c is a perspective view of the Force Test Method equipment.

### DETAILED DESCRIPTION OF THE INVENTION

Section A will provide terms which will assist the reader in best understanding the features of the invention but not to introduce limitations in the terms inconsistent with the context in which they are used in this specification. These definitions are not intended to be limiting. Section B will discuss the pessary applicator of the present invention. Section C will discuss the Force Test Method. Section D will discuss the Length Test Method.

A. Terms

As used herein, the term “pessary” refers to any type of non absorbent vaginal insert for the purpose of reducing urine leakage. Such pessaries may be have any variety of shapes and sizes including cylinders, ovate, spherical, tubular, annual rings, “U” shaped, cup shaped, rings, cubes or donut shaped. They function by direct application of support, level force, expansion of the device by selection of material or by inflation of the device.

As used herein, the terms “vaginal cavity” and “within the vagina” refer to the internal genitalia of the human female in the pudendal region of the body.

As used herein, the term “hymen ring” refers to the demarcation between the vaginal cavity and external genitalia and is identified by the position of the hymen or residual tissue of the hymen.

As used herein, the term “low placement” refers to a position of the pessary inside the vaginal cavity, wherein the base of the pessary is positioned at least about 5 millimeters above the hymen ring and below the cervix.

As used herein, the term “remaining portion” refers to the portion of the pessary which is embedded into the pessary applicator. Specifically, the remaining portion is measured from the most distal portion of the pessary to the most proximal portion of the outer member which includes petals.

As used herein, the term “proximal position” refers to the most proximal position of the pessary positioning member along the longitudinal axis.

The terms “distal” and “proximal,” respectively designated as P and D in the FIGS., herein respectively refer to directions away from and towards the body of the pessary wearer unless otherwise specified.

The term “fully engaged,” as used herein, occurs when the second end of the inner member is flush with the second end of the outer member or after 1250 grams of force has been applied to the end of the inner member.

The term “joined” or “attached” encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element; i.e., one element is essentially part of the other element.

B. Pessary Applicator of the Present Invention

The FIGS. show embodiments of the pessary applicator of the present invention. The present invention, however, is not limited to a structure having the particular configurations shown in the drawings or discussed herein. The pessary applicator of the present invention can be any configuration or size as long as the pessary can be removed from the pessary applicator by the body with a certain amount of force and the pessary applicator expels an exposed portion of the pessary from the pessary applicator. The exposed portion of the pessary may have a larger diameter than the portion of the pessary within the applicator.

In order to better understand the present invention, a detailed description of several non-limiting examples is given below. The below descriptions of the configurations of alternative embodiments are intended to be by way of example and are not intended to limit the invention.

I. Pessary Applicator Having an Outer Member and an Inner Member

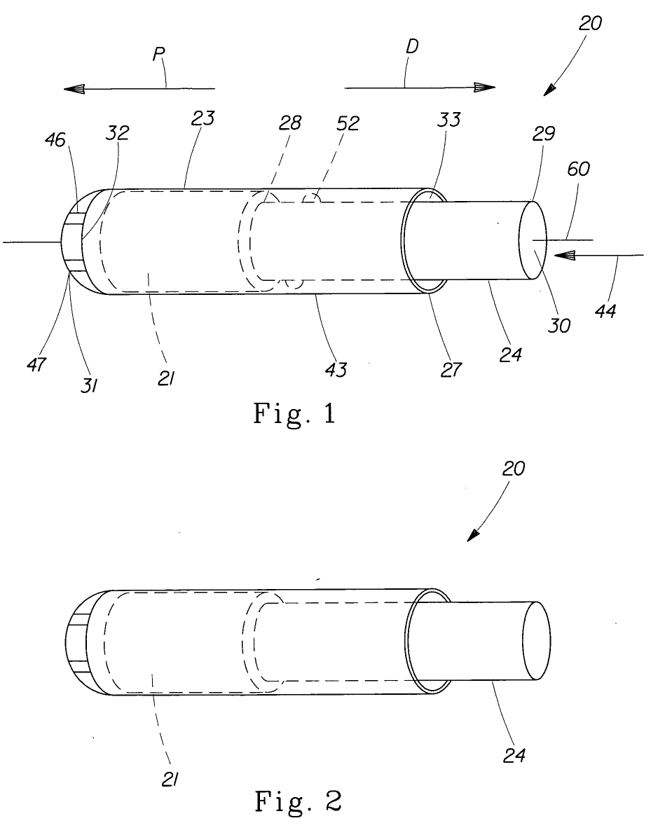

Referring to FIG. 1, a pessary applicator 20 is shown which is designed to position pessary 21 to achieve proper placement inside the vaginal cavity. Specifically, the present invention is directed to a pessary applicator 20 which partially expels a pessary 21. Also, FIG. 1 shows “proximal” and “distal” designated as P and D, respectively. The pessary applicator 20 may be used with any type of pessary 21. The pessary 21 could be a self-sustaining pessary or a deformable pessary.

Generally, the pessary applicator 20 includes an outer member 23 and an inner member 24. The outer member 23 comprises an insertion end 31 and a second end 27 opposed to the insertion end 31. During insertion of the pessary applicator 20 into the body of a wearer, the insertion end 31 is the most proximal end to the body of the wearer along the longitudinal axis 60 and the second end 27 is the most distal end to the body of the wearer along the longitudinal axis 60. A preformed hinge or groove 32 may extend around the periphery of the outer member 23 near the insertion end 31. The outer member 23 may also have a dome-shaped end having a number of radial slits 46 therein extending from a central aperture to the groove 32. The portion of the outer member 23 adjacent to the insertion end 31 may also have an openable end, such as petals 47. In addition, the outer member 23 can contain a grip region 43 located adjacent to the second end 27 of the outer member 23.

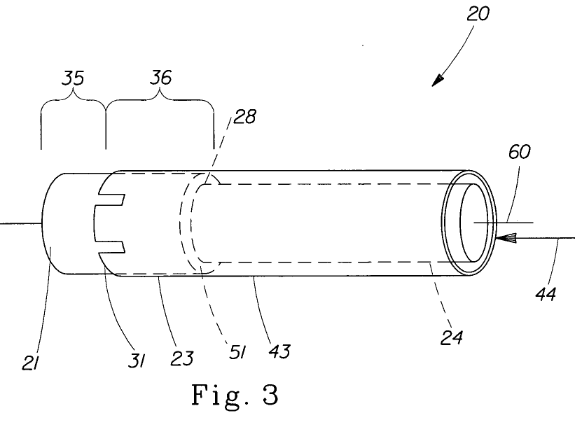

The inner member 24 is dimensioned to slidably move within the hollow interior portion 33 of the outer member 23, with minimal clearance therebetween. The inner member 24 has a first end 28 and a second end 29 opposed to the first end 28. The first end 28 is the most proximal end of the inner member 24 along the longitudinal axis 60. In this embodiment, the first end 28 provides the necessary force to expel the exposed portion 35 (FIG. 3) of pessary 21. In other words, the first end 28 becomes the pessary pushing member 51. The inner member 24 may have nibs 52 located near the first end 28 of inner member 24. The second end 29 is the most distal end of the inner member 24 along the longitudinal axis 60. An axial force 44 is applied to the second end 29 of inner member 24 to expel the exposed portion 35 (FIG. 3) of pessary 21. Also, the inner member 24 can have a hollow interior portion 30. Alternatively, the inner member 24 can be solid or partially solid.

The pessary applicator 20 has a pre-expelled state and a partially expelled state. During the pre-expelled state, as is readily seen in FIG. 1, the pessary 21 sits within the outer member 23 and is substantially aligned with the pessary applicator 20. The pessary 21 can remain snugly therein without any outside force to sustain its position in the pessary applicator 20. In one nonlimiting example, the pessary 21 can be enveloped by the inner member 24 or embedded within inner member 24 during expulsion of pessary 21. The pessary 21 may or may not be in contact with the first end 28 of the inner member 24 before expulsion of the pessary 21. In one nonlimiting example, as seen in FIG. 2, before expulsion of the exposed portion 35 (FIG. 3) of pessary 21 from the pessary applicator 20, the pessary 21 does not contact the inner member 24 and can be substantially aligned with the pessary applicator 20.

As is readily seen in FIG. 3, when the axial force 44 is applied along the longitudinal axis 60, the inner member 24 slides toward the insertion end 31 of the outer member 23. The inner member 24 bears against the rear end of pessary 21 pushing the pessary 21 toward the insertion end 31 of outer member 23. In this embodiment, the first end 28 becomes the pessary pushing member 51. The pessary pushing member 51 provides the necessary force to expel the exposed portion 35 of pessary 21. Upon full engagement of inner member 24 with outer member 23, the exposed portion 35 of the pessary 21 is expelled from the pessary applicator 20 by the pessary pushing member 51 and a remaining portion 36 of the pessary 21 is contained within outer member 23.

During removal of the pessary applicator 20 from the body cavity, the body tissues hold onto the exposed portion 35 of the pessary 21 with the adherence of the pessary surface to vaginal tissues and body pressure. Because the body tissues hold onto the exposed portion 35 of the pessary 21, the pessary applicator 20 exits the body cavity without the pessary 21, i.e., leaving the pessary 21 behind. This results in low placement of the pessary 21 within the vagina.

II. Pessary Applicator Having a Pessary Positioning Member

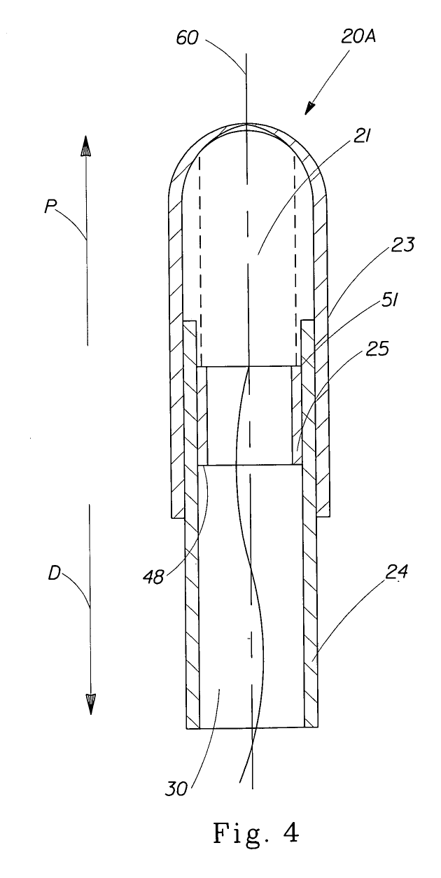

Referring to FIG. 4, in another alternative embodiment, a pessary applicator 20A is shown which is designed to position pessary 21 to achieve proper placement inside the vaginal cavity. Also, FIG. 4 shows “proximal” and “distal” designated as P and D, respectively. Generally, the pessary applicator 20A comprises an outer member 23 and an inner member 24 as discussed in the below section. In addition, the hollow interior 30 of the outer member 23 may contain a pessary positioning member 25.

Referring again to FIG. 4, the pessary positioning member 25 can have a pessary pushing member 51 and a second end 48 opposed to the pessary pushing member 51. The pessary pushing member 51 is the most proximal end of the pessary positioning member 25 along the longitudinal axis 60. The pessary pushing member 51 contacts the pessary 21 and positions the pessary 21 along the length of the inner member 24 so that during expulsion the pessary pushing member 51 forces the pessary 21 to travel a pre-determined distance. This predetermined distance ultimately results in the pessary 21 being positioned properly in the vaginal cavity. The second end 48 is the most distal end of the pessary positioning member 25 along the longitudinal axis 60. In one non-limiting example, the pessary positioning member is a flat surface.

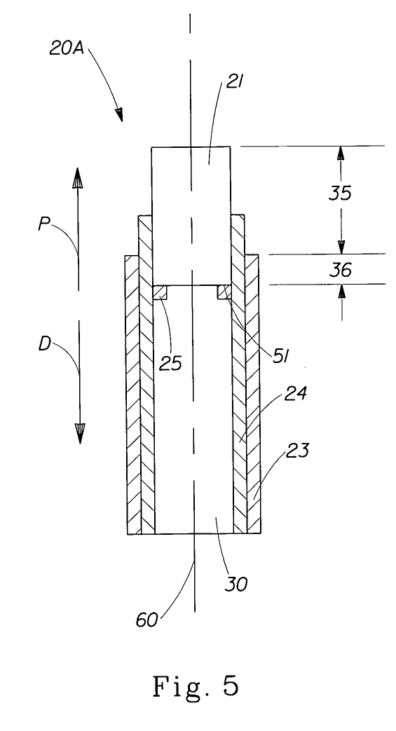

Referring to FIG. 5, in this embodiment, during expulsion of the exposed portion 35 of the pessary 21 from pessary applicator 20A, the remaining portion 36 is embedded in the outer member 23 when the pessary pushing member 51 of the pessary positioning member 25 is in its proximal position and when the pessary applicator 20A has full engagement by having the inner member 24 flush with the outer member 23. Also, FIG. 5 shows “proximal” and “distal” designated as P and D, respectively.

During full engagement of pessary applicator 20A, the pessary pushing member 51 positions pessary 21 such that pessary 21 has an exposed portion 35 expelled from pessary applicator 20A and a remaining portion 36 housed within the inner member 24. During expulsion of the exposed portion 35 of pessary 21 from the pessary applicator 20A, at least a portion of the pessary 21 is adjacent to the pessary pushing member 51 of the pessary positioning member 25. In other words, when the pessary pushing member 51 is at its proximal position along longitudinal axis 60, the pessary pushing member 51 is in contact with the pessary 21 and provides the force necessary to expel the exposed portion 35 of the pessary 21. The body forces inside of the vagina hold onto the exposed portion 35 of the pessary 21 during removal of the pessary applicator 20A from the vaginal cavity.

The pessary positioning member 25 can be fixedly joined to the inner member 24 in any way known in the art. The pessary positioning member 25 can also be monolithic with the inner member 24, for example, by molding the pessary positioning member 25 and the inner member 24 as one piece. In one non-limiting example, the pessary positioning member 25 can be fixedly joined to the inner member 24 by friction fitting that snaps parts together, gluing, and/or melting. In another non-limiting example, the pessary positioning member 25 is attached to the hollow interior portion 30 of the inner member 24. In another non-limiting example, the pessary positioning member 25 can also be a continuation of the inner member 24 by folding or compressing a portion of the inner member 24 and turning the inner member 24 inwards upon itself to create a tube inside a tube.

It is further noted herein that the shape of the pessary positioning member 25 can vary as long as the pessary positioning member 25 aids in positioning the pessary 21 along the length of the inner member 24 to provide proper placement of the pessary 21 within the vaginal cavity. In one embodiment, the pessary positioning member includes an elongated pessary positioning member of a generally tubular shape. Alternatively, the pessary positioning member can be a rod. In another embodiment, the pessary positioning member can be slideable within the outer member 23. In another alternative embodiment, the pessary positioning member can also comprise projections extending inward from the hollow interior 30 of the inner member 24. The projections can be of any shape or size as long as the projections allow the pessary 21 to be in contact with the projections during the expulsion. The projections can be fixedly joined to the inner member 24 in any way known in the art.

It is further noted herein that the length of the pessary positioning member 25 can vary as long as the pessary positioning member 25 aids the pessary 21 to be located in a position within the inner member 24 that will provide proper placement within the vaginal cavity. In one non-limiting example, the length of the pessary positioning member 25 can be from about 36 millimeters to about 63 millimeters.

III. Inner Member Embedded into Pessary

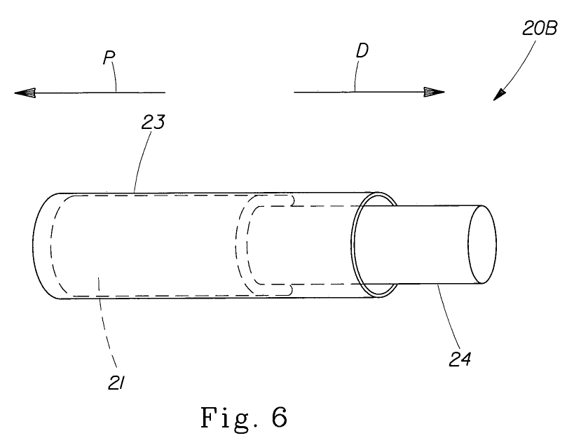

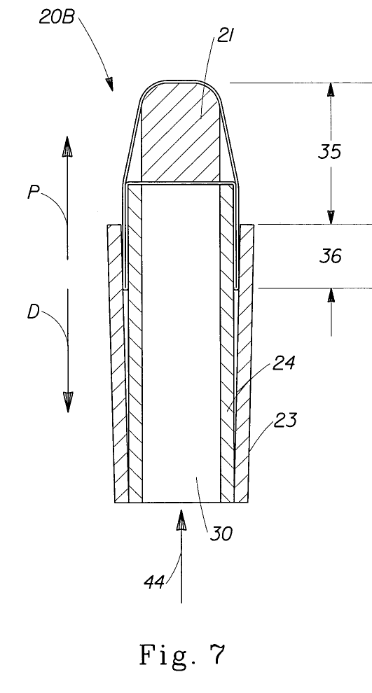

Referring to FIG. 6, in yet another alternative embodiment, a pessary applicator 20B is shown before expulsion of the exposed portion 35 (FIG. 7) of pessary 21. Pessary applicator 20B is designed to position pessary 21 to achieve proper placement inside the vaginal cavity. Also, FIG. 6 shows “proximal” and “distal” designated as P and D, respectively. In this non-limiting example, generally, the pessary applicator 20B comprises an outer member 23 and an inner member 24. In this alternative embodiment, however, the inner member 24 can be embedded within the pessary 21 upon full engagement of the pessary applicator 20B. In other words, the inner member 24 can penetrate at least a portion of the base of the pessary 21 upon full engagement of the pessary applicator 20B.

Referring to FIG. 7, upon full engagement of the outer member 23 with the inner member 24, an exposed portion 35 is expelled from the pessary applicator 20B and a remaining portion 36 of the pessary 21 is contained within pessary applicator 20B. Also, FIG. 7 shows “proximal” and “distal” designated as P and D, respectively. In addition, upon full engagement at least a portion of the inner member 24 is adjacent to the pessary 21. When the inner member 24 is embedded into pessary 21, the inner member 24 provides the force 44 to expel the exposed portion 35 of pessary 21 from pessary applicator 20B.

IV. Examples of the Pessary Applicator of the Present Invention

Examples 1-13, provided below, are embodiments of the pessary applicator of the present invention which expels a percentage of the pessary (i.e. an exposed portion) upon full engagement of the pessary applicator of the present invention. Also, a certain amount of force is required to remove the remaining portion (i.e. embedded portion) of the pessary from the applicator during removal of the pessary applicator from the vaginal cavity. The present invention is not limited to the above structures having the particular configurations shown in the drawings or discussed above herein. Thus, the pessary applicator can be any configuration or size.

| | Percentage of the Length of | Force (N) Required to |
|---|---|---|
| | the Pessary which is | Remove the Remaining |
| | Remaining (i.e. embedded) | Portion of the Pessary from |
| | in the Pessary Applicator | the Pessary Applicator |
| Example | upon Full Engagement | upon Full Engagement |
| Example 1 | About 15% | Less than about .30 |
| Example 2 | About 15% | Less than about .25 |
| Example 3 | About 15% | Less than about .20 |
| Example 4 | About 15% | Less than about .15 |
| Example 5 | About 15% | Less than about .10 |
| Example 6 | About 30% | Less than about .30 |
| Example 7 | About 35% | Less than about .30 |
| Example 8 | About 20% | Less than about .50 |
| Example 9 | About 20% | Less than about .40 |
| Example 10 | About 20% | Less than about .30 |
| Example 11 | About 20% | Less than about .20 |
| Example 12 | About 30% | Less than about .50 |
| Example 13 | About 35% | Less than about .50 |

Examples 14-20, provided below, are embodiments of the pessary applicator of the present invention in which a remaining portion of the pessary (measured in mm) is embedded within the outer member upon full engagement of the inner member with the outer member. Also, a certain amount of force is required to remove the remaining portion of the pessary from the pessary applicator during removal of the pessary applicator from the vaginal cavity. The present invention is not limited to the above structures having the particular configurations shown in the drawings or discussed above herein. Thus, the pessary applicator can be any configuration or size.

| | Length of the pessary | Force (N) Required to |
|---|---|---|
| | which is Remaining (i.e. | Remove the Remaining |
| | embedded) within the | Portion of the Pessary from |
| | Outer Member upon Full | the Pessary Applicator |
| Example | Engagement (mm) | upon Full Engagement |
| Example 14 | About 12 | Less than about 0.50 |
| Example 15 | About 13 | Less than about 0.50 |
| Example 16 | About 15 | Less than about 0.50 |
| Example 17 | About 8 | Less than about 0.35 |
| Example 18 | About 10 | Less than about 0.35 |
| Example 19 | About 10 | Less than about 0.30 |
| Example 20 | About 12 | Less than about 0.40 |

Examples 21-24, provided below, are embodiments of the pessary applicator of the present invention which expels an exposed portion of the pessary upon full engagement of the pessary applicator of the present invention. The present invention requires a certain amount of force per millimeter to remove the pessary from the pessary applicator of the present invention. The present invention, however, is not limited to the above structures having the particular configurations shown in the drawings or discussed above herein. Thus, the pessary applicator can be any configuration or size.

| | Force (N/mm) Required to Remove the Remaining |
|---|---|
| | Portion (i.e. Embedded Portion) of the Pessary |
| Example | from the Pessary Applicator upon Full Engagement |
| Example 21 | Less than about 0.04 |
| Example 22 | Less than about 0.03 |
| Example 23 | Less than about 0.02 |
| Example 24 | Less than about 0.04 |

Examples 25-26, provided below, are embodiments of the pessary applicator of the present invention in which a percentage of the pessary remains embedded within the outer member upon full engagement of the inner member with the outer member. Also, a certain amount of force per millimeter is required to remove the remaining portion of the pessary from the pessary applicator during removal of the pessary applicator from the vaginal cavity. The present invention is not limited to the above structures having the particular configurations shown in the drawings or discussed above herein. Thus, the pessary applicator can be any configuration or size.

| | | Force (N/mm) Required to |
|---|---|---|
| | | Remove the Remaining |
| | Percentage of the Length of | Portion (i.e. Embedded |
| | the Pessary which is | Portion) of the Pessary |
| | Remaining (i.e. embedded) | from the Pessary |
| | in the Pessary Applicator | Applicator upon Full |
| Example | upon Full Engagement | Engagement |
| Example 25 | About 10% | Less than about 0.04 |
| Example 26 | About 12% | Less than about 0.03 |

Below will provide more detail of each component of the pessary applicator of the present invention.

a. Outer Member

Referring to FIG. 1, typically, the outer member 23 can be used to handle or grip the pessary applicator 20 during the insertion into the vaginal cavity. The outer member 23 is external to the inner member 24. The outer member 23 has a hollow interior 33, an insertion end 31, and a second end 27 opposed to the insertion end 31. The insertion end 31 is the most proximal end of the outer member 23. In addition, the insertion end 31 is the first portion of the outer member 23 which is inserted into the vaginal cavity and it is the end from which the pessary 21 is expelled. The second end 27 is the most distal portion of the outer member 23 along the longitudinal axis 60. Grip region 43 is adjacent to the second end 27. Grip region 43 is the portion of the outer member 23 in which the user can handle or grip the pessary applicator 20. At least a portion of the hollow interior 33 of the outer member 23 can engage with at least a portion of the inner member 24. In one non-limiting example, the inner member 24 can be slideable within the outer member 23.

The manufacturer of the pessary applicator 20 can vary the size of the outer member 23. The size of the outer member 23 can be determined primarily by the dimensions of the pessary 21. Specifically, the diameter of the outer member 23 can be varied to accommodate different absorbency pessarys. Generally, the outer member 23 can have an inner diameter of from about 6 millimeters to about 21 millimeters and a wall thickness of from about 0.4 millimeter to about 1.2 millimeter. Generally, the inner diameter of the outer member 23 should be suitably greater than the diameter of the pessary 21 to prevent the outer member 23 from interfering with the removal of the pessary 21 from the outer member 23. Moreover, the inner diameter of the outer member 23 can have varying diameters and shapes to conform to the profiled shape of the enclosed pessary 21.

Also, the manufacturer of the pessary applicator 20 can vary the length of the outer member 23. Generally, the outer member 23 should be of a sufficient length to house at least a portion of the pessary 21 and preferably all of pessary 21 prior to the expulsion of the pessary 21 from the pessary applicator 20 into the vaginal cavity. Generally, the length of the outer member 23 can be from about 50 millimeters to about 100 millimeters. The length of the outer member 23 is preferably from about 50 mm to about 90 mm, more preferably from about 55 mm to about 85 mm.

In addition, the manufacturer of the pessary applicator 20 can vary the shape of the outer member 23. It is further noted herein that the shape of the outer member 23 can vary as long as a portion of the inner member 24 can be slideable within the outer member 23. In one non-limiting example, the grip region 43 can be of a smaller perimeter than the remainder of the outer member 23. In this non-limiting example, the inner member 24 can be a rod which slidably moves within the hollow interior portion 33 of the outer member 23. Moreover, the outer member 23 is in no way limited by the shape that it can assume except that the shape should not hinder directional expulsion of the pessary 21. One skilled in the art can imagine that the outer member 23 might be cylindrical, ovate or curved like a banana or any other suitable shape as long as any such shape would work effectively to allow pessary 21 to expel from the pessary applicator 20, and comfortably insert into the vaginal cavity. The outer member 23 can be of any suitable cross-sectional shape. For example, suitable cross-sectional shapes can include, but are not limited to, circular, oval, flattened circular, elliptical, and any combination thereof.

Furthermore, the outer member 23 can contain the grip region 43 as noted above. The grip region 43 is located between the insertion end 31 and the second end 27 and closer to the second end 27 of the outer member 23. The grip region 43 can provide for secure handling of the outer member 23. The perimeter of the grip region 43 can take essentially any desired shape, including oval, circular, and various other geometric forms. The gripping region 43 can be defined by outward projections or raised surfaces, created by impressing or compressing the surfaces. Referring again to FIG. 1, the grip region 43 is substantially circularly shaped, but it can also take on more angular formations such as squared. The surface of the grip region 43 can be any kind of surface known in the art. This surface can provide a desired frictional resistance for the fingers during the insertion of the pessary applicator 20 into the body. Moreover, this surface area can have, for example, a dove pattern created via surface texturing, impressions, and/or indentations.

Furthermore, it is preferable that the grip region 43 be of sufficient dimension to substantially provide a comfortable grip for the user. As used in the specification, the term “user's grip” means any way of holding the pessary applicator 20 in a hand, e.g., between a thumb and a finger. The grip region 43 need not necessarily extend completely around the perimeter of the outer member 23. For example, a space can be provided for a decorative marking or a Trademark character.

b. Inner Member

In FIG. 1, in some embodiments of the present invention, inner member 24 can be designed to store at least a portion of the pessary 21 within its hollow interior 30 either during the expulsion and/or before the expulsion of the pessary 21 from the insertion end 26 of the outer member 23. The inner member 24 has a hollow interior 30. Alternatively, the inner member 24 can be solid or partially solid. The first end 28 is the most proximal end of the inner member 24 along the longitudinal axis 60. The second end 29 is the most distal end of the pessary applicator 20 along the longitudinal axis 60. Moreover, the second end 29 is the portion of the inner member 24 in which the axial force 44 is applied to expel the pessary 21 from the outer member 23.

The manufacturer of the pessary applicator 20 can vary the shape of the inner member 24 as long as any such shape would work effectively to properly aide in the expulsion of the pessary 21 from the pessary applicator 20 and comfortably insert into the vaginal cavity. One skilled in the art can imagine other shapes of the inner member 24, for example, cylindrical or curved like a banana or any other suitable shape possible. For example, the inner member 24 can be of any suitable cross-sectional shape including, but are not limited to, circular, oval, flattened circular, elliptical, and any combinations thereof.

The size of the inner member 24 can be determined primarily by the dimensions of pessary 21. Specifically, the diameter of the inner member 24 can be varied to accommodate different absorbency pessarys. For example, when pessary 21 is housed within the inner member 24, higher protecting pessarys can have larger diameters resulting in the inner diameter of the inner member 24. In addition, the inner diameter of the inner member 24 can have varying diameters and shapes to conform to the profiled shape of the enclosed pessary 21.

The length of the inner member 24 can also vary. If the pessary 21 is housed within inner member 24, generally, the inner member 24 should be of a sufficient length to house a substantial portion of the pessary 21 prior to the insertion of the applicator into the body, thus aiding in the proper expulsion of the pessary 21 from the pessary applicator 20, and comfortably inserting into the vaginal cavity. For example, the length of the inner member 24 can be from about 45 millimeters to about 90 millimeters.

Optionally, it can be seen that inner member 24 can have nibs 52 provided on the perimeter thereof located near the first end 28 of inner member 24. If nibs 52 are included, the inner member 24 should fit within the outer member 23. The nibs 52 can be adapted to engage with the optional grooves on the inside surface (not shown) of outer member 23 thereby providing a number of possible predetermined relative positions for the outer member 23 and for the inner member 24. Nibs 52 can prevent the inner member 24 from too easily being pushed into the outer member 23 (resulting in premature expulsion of the pessary 21) should the pessary applicator 20 be mishandled before the use thereof. Nibs may also be used to stop the movement of the inner member 24 to signal full engagement of the inner member 24 and the outer member 23.

c. Pessary Applicator Materials

Different pessary applicator parts can be constructed from different materials and processes. The pessary applicator or any part of the pessary applicator can be formed of a spirally wound, convolutedly wound, or longitudinally seamed hollow tube that is formed from paper, paperboard, cardboard, or any combinations thereof. The pessary applicator or any part of the pessary applicator can also be injection molded, extruded, or formed from flexible plastic, such as thermoformed from plastic sheet or folded or wound from plastic film.

The pessary applicator or any part of the pessary applicator can be constructed from a single ply of material or be formed from two or more plies that are bonded together to form a laminate. The use of two or more plies or layers is preferred for it enables the manufacturer to use certain materials in the various layers that can enhance the performance of the pessary applicator or any part of the pessary applicator. When two or more plies are utilized, all the plies can be spirally wound, convolutedly wound, or longitudinally seamed to form an elongated cylinder. The pessary applicator or any part of the pessary applicator can be constructed using a smooth thin ply of material on the outside or exterior surface that surrounds a coarser and possibly thicker ply. When the pessary applicator or any part of the pessary applicator contains at least three plies, the middle ply can be the thicker ply, and the interior and exterior plies can be smooth and/or slippery to facilitate the expulsion of the pessary and to facilitate the insertion of the pessary applicator or any part of the pessary applicator into a woman's vagina. By sandwiching a thick, coarser ply of material between two thin, smooth plies, a pessary applicator or any part of the pessary applicator can be provided which is very functional. The pessary applicator or any part of the pessary applicator should contain one to four plies, although more plies can be utilized if desired.

The plies forming the pessary applicator or any part of the pessary applicator can be held together by an adhesive, such as glue, heat, pressure, ultrasonic, or any combinations thereof. The adhesive can be either water-soluble or water-insoluble. A water-soluble adhesive is preferred for environmental reasons in that the pessary applicator or any part of the pessary applicator will quickly break apart when it is immersed in water. Such immersion will occur should the pessary applicator or any part of the pessary applicator be disposed of by flushing it down a toilet. Exposure of the pessary applicator or any part of the pessary applicator to a municipal's waste treatment plant wherein soaking in water, interaction with chemicals, and agitation all occur, will cause the pessary applicator or any part of the pessary applicator to break apart and evenly disperse in a relatively short period of time.

C. Force Test Method

i. Overview

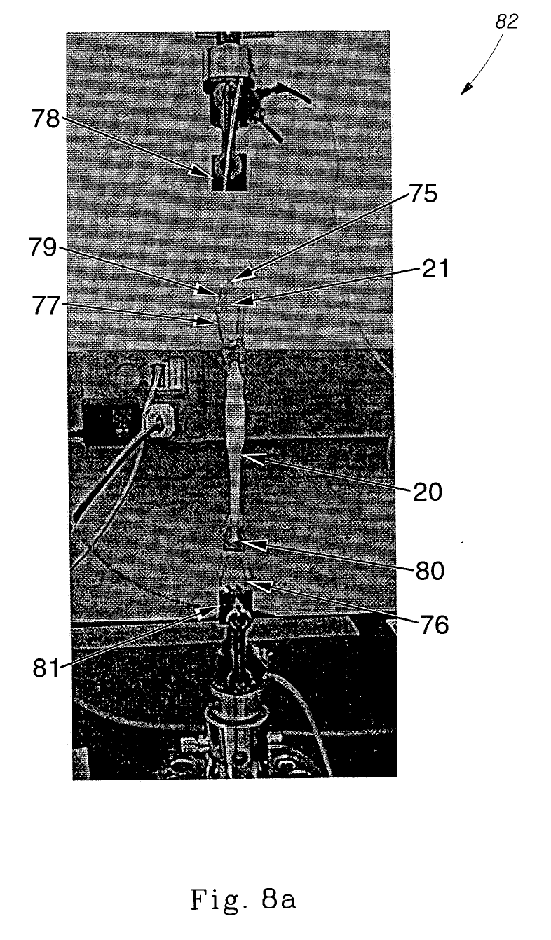

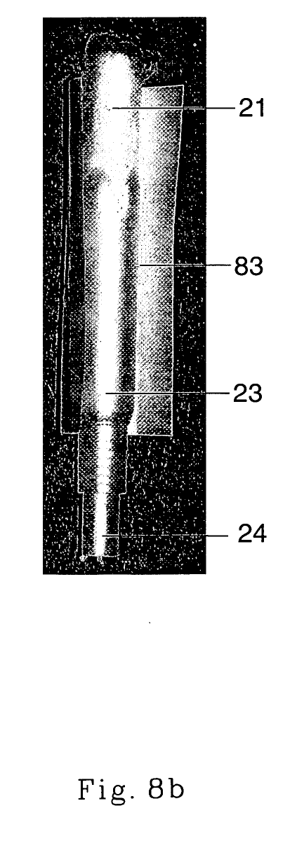

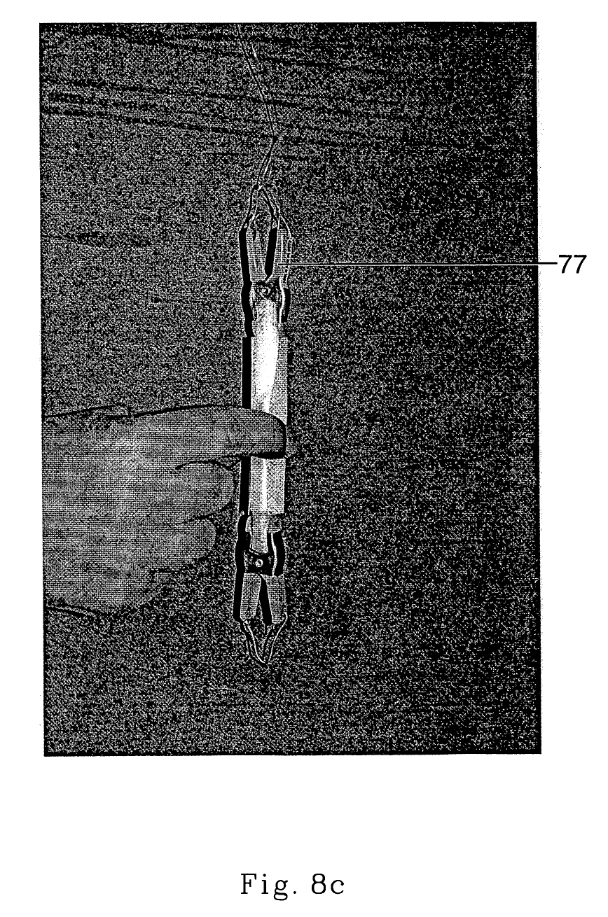

Referring to FIG. 8a, this test is intended to measure the amount of force that is required to remove the remaining portion of the pessary from a pessary applicator which is fully engaged.

The less length of the pessary 21 exposed after expulsion from the pessary applicator, the lower the pessary 21 is placed in the vaginal cavity when compared to an identical pessary-applicator system where the pessary is fully expelled. When the pessary 21 is placed low in the vaginal cavity there is better support of the bladder and urethra because a pessary 21 placed at the appropriate depth in the vagina is more effective in providing support of the cervix and pressure against the urethra when placed in the center and in front of the cervix.

ii. Test Stand Preparation

The Force Removal Test uses an Instron Model 5564 with a 10 Newton load cell, which is available from Instron Corporation of Canton, Mass.; standard 1.0 inch grips operated at 60 psi were used. The test stand also uses two ¾″ coated plastic tip mini-Spring Clamps (model number 614-593 WorkForce, 2455 Paces Ferry Rd, Atlanta, Ga. 30339 purchased from Home Depot (#3579462622 8)); 0.0198 inch diameter fishing line manufactured by Shakespeare Model WP550; and a folded wire paper clip (no. 1 Gem Clip, Model XP40020).

Machinery Settings: The Instron crosshead speed is set at 508 mm/min. The force on the fishing line 79 is adjusted to +/−5 grams so that it sets a standard initial force on the pessary 21 and eliminates significant excess slack in the fishing line 79. The cross head travel is the distance required for the pessary 21 to be fully removed from the pessary applicator 20. Data is collected at 100 Hz starting at 2 mm of cross head extension. The peak force of the test is the maximum force in Newtons obtained as the pessary 21 is removed from the pessary applicator 20 minus the Baseline Force contributed by the test pessary, upper utility clip assembly, and fishing line. The Baseline Force may be determined by the force applied to the Instron after the pessary 21 is totally removed from the pessary applicator. Ten pessary-applicator assemblies are analyzed and the average force is reported.

The following steps occur to prepare the test stand 82 as shown in FIG. 8a:
1. Gem Clips: Unfold the Gem Clips 75, 76 until the Gem Clips 75, 76 form a substantially straight line. Form the clips into a “U” or “V” shape.
2. Upper Utility Clip Assembly: Run both ends of the first Gem Clip 75 through the holes at the base of the upper mini-Spring Clamp 77 so that the U portion of the Gem Clip 75 is facing away from the mini-Spring Clamp 77. The Gem Clip 75 is secured to the mini-Spring Clamp 77 by bending and twisting it around itself. Secure a first end of the 12 inch section of fishing line 79 to the top of the center U of the first Gem Clip 75.
3. Lower Utility Clip Assembly: Run the ends of the second Gem clip 76 through the holes at the base of lower mini-Spring Clamp 80. Form the second Gem paper clip 76 into a substantially U-shaped configuration and secure the second Gem clip 76 to the mini-Spring Clamp 77 by bending and twisting it around itself.
4. Prepare Stabilizing Fixture. Referring to FIG. 8b, a Stabilizing Fixture 83 may be required to stabilize the fully plunged pessary-applicator system 20 as it is placed into the test stand 82 (FIG. 8a). The stabilizing fixture 83 is a flexible partial tube made from paper manila letter size File Folders obtained from The Smead Manufacturing Co., Hastings, Mn. Part number 135L, UPC No. 10330. The paper folder is sized such that when the inner member 24 is fully engaged the pessary extends about 1 cm from the longitudinal edge and its width is from about 1 mm to about 5 mm less than the outer member 23 of the pessary applicator 20. The paper is then rolled along the longitudinal axis into a partial tube that can be used to hold the pessary-applicator assembly together. While the Stabilizing Fixture 83 may be adjusted to the specific dimensions of the pessary applicator 20, typical dimensions are about 85 mm long and about 50 mm in width prior to forming the tube.
5.

ii. Sample Preparation

To prepare the sample pessary for use in the Force Test Method, the withdrawal string of the pessary must be trimmed so that it is flush with the inner member. The withdrawal string is trimmed to prevent it from catching a surface when the pessary is extracted from the pessary applicator during any inner member movement, or during the Instron test.

Referring to FIG. 8a, the following must be done to prepare the samples prior to testing:

1. The pessary applicator samples are removed from the box and equilibrated at 23° C.±1° C. and at a relative humidity of 50%±2% for 2 hours within the applicator and wrapper, if the pessarys are individually wrapped.
2. Remove the pessary from the wrapper (if present) and complete the sample preparation and testing within 10 minutes after removal from the wrapper.
3. Determination of Plunge length. Plunge the pessary applicator 20 by pushing the inner member 24 against a solid flat surface until the inner member is fully engaged. A full inner member 24 engagement with outer member 23 is completed when 1250 g of force is applied to the inner member 24. If the pessary 21 is not retained in the pessary applicator 20 or retained by less than 2 mm, the sample is outside the limits of this test as determined by the Force Test Method.
   a. Place the pessary applicator assembly horizontally onto the Stabilizing Fixture 83 so that when the pessary 21 is deployed the leading edge of the pessary extends about 1 cm from longitudinal edge of one end of the Stabilizing Fixture 83. The grip zone of the applicator 20 should extend about 1 cm from the opposite end of the Stabilizing Fixture 83.
   b. Grasp the grip zone of the pessary applicator 20 and plunge the pessary applicator 20 to the fully engaged position.
   c. Carefully grasp the side of the Stabilizing Fixture 83 about 1 cm below the proximal end of the pessary applicator 20, applying only sufficient pressure to hold the pessary-applicator system 20 in a stable configuration.
   d. Referring to FIG. 8c, attach the Upper Utility Clip Assembly 77 to the top 1 cm of the proximal end of the pessary 21.
   e. Referring to FIG. 8a, attach the Lower Utility Clip Assembly 80 to the finger grip of the pessary applicator 20. While carefully holding the pessary-applicator system in the Stabilizing Fixture 83 (FIG. 8b), seat the concave portion of the substantially u-shaped configuration of the GEM clip of the Lower Utility Clip Assembly 76 into the lower Instron grip 81. This will allow the Lower Utility Clip Assembly 76 to be anchored in the lower Instron grip 81 of the test stand 82.
   f. Insert the fishing line of the Upper Utility Clip Assembly 77 into the upper Instron grip 78. Carefully remove the Stabilizing Fixture 83 (FIG. 8b) from around the pessary-applicator assembly.
   g. If the pessary is displaced or misaligned during the assembly or during the attachment between the Instron grips 81, discard the sample and repeat the procedure.

Applicators of the present invention will preferably have an average net peak load measured in Newtons of about 0.036. The clip, line, & pessary weight are subtracted from the net peak load. During removal of the pessary applicator from the body cavity, less force is required to remove the pessary from the pessary applicator 21 so that body tissues can hold onto the pessary 21. The surface texture of the pessary can be modified to enhance the retention of the device within the body, or the pessary may be covered with a slightly absorbent overwrap to enhance the ability of the vagina to retain the pessary. Since the pessary is not fully expelled from the pessary applicator, the body tissues are required to hold onto the pessary 21, extracting it from the pessary applicator as it exits the body cavity without the pessary 21, i.e., leaving the pessary 21 behind. This results in low placement of the pessary 21 within the vagina compared to pessarys fully expelled from applicators of similar length.

D. Length Test Method

i. Overview

This test defines the amount of pessary embedded in the pessary applicator of the present invention to provide low placement in the vaginal cavity compared to identical pessary applicator systems without the pessary embedded. This is done by determining the length of the pessary exposed from the pessary applicator and the length of the pessary embedded in the below-mentioned pessary applicator samples. The less length of the pessary exposed after fully engaging the pessary applicator, the lower the pessary 21 is placed in the vaginal cavity when compared to identical pessary-applicator systems without the pessary embedded. When the pessary 21 is placed low in the vaginal cavity there is greater coverage in the vaginal cavity because a pessary 21 placed lower in the vagina is more effective in covering the vagina given that the vagina is typically narrower in the lower region and the pessary is placed in the center and front of the cervix.

ii. Sample Preparation

The following must be done to prepare the samples prior to testing:

1. The pessary applicator samples are removed from the box and equilibrated at 23° C.±1° C. and at a relative humidity of 50%±2% for 2 hours within the applicator and wrapper for the pessary applicator, if pessarys are individually wrapped.
2. Remove the pessary from the wrapper (if present) and complete the sample preparation and testing within 10 minutes after removal from the wrapper.

a. Plunge Type Applicators
i. Remove the pessary applicator from the wrapper, if present.
ii. Fully Engaged Position: Plunge the applicator with 1250 grams of force applied to the inner member. This is the fully engaged position. The Fully Engaged Position is the average from 10 pessary-applicators systems.
iii. Cut approximately 7 cm of 0.75 inch wide Highland Invisible Tape (3M, Stationery Products Division, St. Paul, Minn. 55119).
iv. Place the Invisible Tape on a smooth flat surface with the adhesive side facing upward.
v. Place the pessary applicator system on the tape perpendicular to the length of the tape.
vi. The proximal end of the pessary applicator should center on the 0.75 in. width of the tape. The pessary applicator should also be placed on one end of the tape such that the tape is positioned directly beneath the pessary applicator so excess tape does not interfere with the formation of the cylinder in step vii.
vii. Roll the pessary applicator across the tape so the entire length of tape forms a cylinder around the proximal end of the pessary applicator.
viii. While the pessary applicator is flat on the table, grasp the distal end of the pessary applicator at the finger grip, press the inner member to the Fully Engaged Position to expel the pessary. Inspect the expelled pessary-applicator system to insure the pessary is aligned along the longitudinal axis 60.
ix. Cut the pessary removal cord flush with the distal end of the applicator.
x. Length of Fully Engaged Pessary-Applicator System, L1.
   a. Adjust the vertical height of a digital caliper gauge (Ono-Sokki Linear Gauge Sensor GS-503 with a GS-3610 digital reader) so that it can accommodate an applicator-pessary assembly of the appropriate length. Alternatively, if the pessary-applicator assembly is too long to be accommodated, rotate the gauge so the table top surface becomes the effective base. The diameter of the calipers foot is 24.2 mm and force applied by the foot is 0.32N.
   b. Orientate the pessary applicator assembly so the longitudinal axis is perpendicular to the caliper gauge base with the proximal end of the pessary pointing upward to the caliper gauge foot and firmly press the distal end of the fully engaged applicator against the caliper gauge base. Care must be taken to insure the pessary remains in the vertical position during all measurements. If the pessary-applicator system orientation shifts relative to the gauge during measurements, disregard the measurement. If the pessary permanently shifts relative to the pessary applicator during measurement, for example, the pessary slips downward or off center or the pessary applicator petals slip under the pessary, disregard the sample and repeat.
   c. Make five measurements on each pessary-applicator system and repeat with 10 pessarys-applicator systems. While the pessary applicator is in its fully engaged position, the distance as measured from the most distal end of the pessary applicator to the most proximal end of the pessary is recorded as L1.
   d. Report the average L1 of all measurements.
xi. Length of Fully Engaged Applicator, L2.
   a. Using the afore-mentioned samples to measure L1, measure the distance from the most distal end of the pessary applicator to the most proximal end of the outer member in the pessary applicator in its fully engaged position with a hand-held electronic digital caliper gauge (VWR Digital Caliper, 15.2 cm 6″ caliper, Catalog Number 62379-531). If the pessary applicator has petals, the distal end of the pessary applicator is the tip of the petals extended by the pessary.
   b. Measure at least three petals approximately equally spaced around the pessary applicator, report the average. If any petals have folded under the pessary, that sample should be discarded and a new sample selected. If a pessary shows evidence of slippage or change of orientation relative to the pessary applicator, discard the sample and a new sample is selected. The distance L2 is recorded as the average from 10 pessarys.
xii. Pessary Length, L3.
   a. Using a second set of pessarys, carefully remove the pessary 21 from the pessary applicator.
   b. Measure the length in mm of the main body of the pessary 21, for typical pessarys this is the rigid body of the pessary. The main body of the pessary does not include any overwrap, secondary absorbent member, or withdrawal cord which extends beyond the pessary's main body
   c. Carefully cut the removal cord from the body of the pessary including any overwrap, secondary absorbent member, or withdrawal cord which extends beyond the pessary's main absorbent material.
   d. Adjust the vertical height of a digital caliper gauge (Ono-Sokki Linear Gauge Sensor GS-503 with a GS-3610 digital read) above its base so that it can accommodate a pessary of the appropriate length. The diameter of the calipers foot is 24.2 mm and force applied by the foot is 0.32 N. Orientate the pessary so the longitudinal axis is perpendicular to the caliper gauge base with the proximal end of the pessary pointing upward to the caliper gauge foot and the distal end oriented towards the base of the gauge. Care must be taken to insure the pessary is in the vertical position during all measurements, if the pessary orientation shifts during measurements disregard the measurement.
   e. Make five measurements on each pessary and repeat with 10 pessarys. The distance as measured from the distal end of the pessary to the proximal end of the pessary is as L3.
xiii. The Embedded Pessary Length is L3 plus L2 minus L1, reported in mm.
xiv. The force (N) per mm of embedded pessary is the Pessary Removal Force divided Embedded Pessary Length in mm.
xv. The Embedded Length Method may be modified to accommodate non-traditional-pessary applicator systems so that the pessary is exposed from the applicator consistent with the applicator design.

Pessary applicators of the present invention preferably expose less length of the pessary 21 and embed more of the pessary within the inner member. Accordingly, the less the pessary is exposed post insertion and the more the pessary is embedded, the lower the placement of the pessary within the vaginal cavity. During removal of the pessary applicator from the body cavity, the body tissues hold onto the pessary 21 with the adherence of the pessary surface to vaginal tissues and body pressure. Because the body tissues hold onto the pessary 21, the pessary applicator exits the body cavity without the pessary 21, i.e., leaving the pessary 21 behind. This results in lower placement of the pessary 21 within the vagina when compared to an identical pessary-applicator system where the pessary is not embedded.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A pessary applicator for positioning a pessary inside a vaginal cavity, said applicator comprising:
i. an outer member disposed co-axially with an inner member for insertion of said pessary into said vaginal cavity;
ii. said inner member being slidable within said outer member
wherein at least 15% of a length of said pessary remains in said outer member when said inner member is fully engaged with said outer member to define a remaining portion of said pessary, said remaining portion of said pessary requiring a force of less than about 0.30 N to be removed from said pessary applicator.

2. The pessary applicator according to claim 1 wherein said remaining portion of said pessary requires a force of less than about 0.30 N to be removed from said outer member.

3. The pessary applicator according to claim 1 wherein said remaining portion of said pessary requires a force of less than about 0.30 N to be removed from said inner member.

4. The pessary applicator according to claim 1 wherein said remaining portion of said pessary requires a force of less than about 0.25 N to be removed from said outer member.

5. The pessary applicator according to claim 1 wherein said remaining portion of said pessary requires a force of less than about 0.20 N to be removed from said outer member.

6. The pessary applicator according to claim 1 wherein said remaining portion of said pessary requires a force of less than about 0.15 N to be removed from said outer member.

7. The pessary applicator according to claim 1 wherein said remaining portion of said pessary requires a force of less than about 0.10 N to be removed from said outer member.

8. The pessary applicator according to claim 1 wherein at least 30% of said pessary length remains in said outer member when said inner member is fully engaged with said outer member.

9. The pessary applicator according to claim 1 wherein at least 35% of said pessary length remains in said outer member when said inner member is fully engaged with said outer member.

10. A pessary applicator for positioning a pessary inside a vaginal cavity, said applicator comprising:
i. an outer member disposed co-axially with an inner member for insertion of said pessary into said vaginal cavity;
ii. said inner member being slidable within said outer member
wherein at least 20% of a length of said pessary remains in said outer member when said inner member is fully engaged with said outer member to define a remaining portion of said pessary, said remaining portion of said pessary requiring a force of less than about 0.50 N to be removed from said outer member.

11. The pessary applicator according to claim 10 wherein said remaining portion of said pessary requires a force of less than about 0.40 N to be removed from said outer member.

12. The pessary applicator according to claim 10 wherein said remaining portion of said pessary requires a force of less than about 0.30 N to be removed from said outer member.

13. The pessary applicator according to claim 10 wherein said remaining portion of said pessary requires a force of less than about 0.20 N to be removed from said outer member.

14. The pessary applicator according to claim 10 wherein at least 30% of said pessary length remains in said outer member when said inner member is fully engaged with said outer member.

15. The pessary applicator according to claim 10 wherein at least 35% of said pessary length remains in said outer member when said inner member is fully engaged with said outer member.

16. The pessary applicator of claim 10 wherein at least 12 mm of said pessary remains in said outer member when said inner member is fully engaged with said outer member.

17. The pessary applicator of claim 10 wherein at least 13 mm of said pessary remains in said outer member when said inner member is fully engaged with said outer member.

18. The pessary applicator of claim 10 wherein at least 15 mm of said pessary remains in said outer member when said inner member is fully engaged with said outer member.

19. A pessary applicator for positioning a pessary inside a vaginal cavity, said applicator comprising:
i. an outer member disposed co-axially with an inner member for insertion of said pessary into said vaginal cavity;
ii. said inner member being slidable within said outer member
wherein a length of at least 8 mm of said pessary remains in said outer member when said inner member is fully engaged with said outer member to define a remaining portion of said pessary, said remaining portion of said pessary requiring a force of less than about 0.35 N to be removed from said outer member.

20. The pessary applicator of claim 19 wherein at least 10 mm of said pessary remains in said outer member when said inner member is fully engaged with said outer member and said remaining portion of said pessary requires a force of less than about 0.35 N to be removed from said outer member.

21. The pessary applicator of claim 19 wherein at least 10 mm of said pessary remains in said outer member when said inner member is fully engaged with said outer member and said remaining portion of said pessary requires a force of less than about 0.30 N to be removed from said outer member.

22. The pessary applicator of claim 19 wherein at least 12 mm of said pessary remains in said outer member when said inner member is fully engaged with said outer member and said remaining portion of said pessary requires a force of less than about 0.40 N to be removed from said outer member.

23. A pessary applicator for positioning a pessary inside a vaginal cavity, said applicator comprising:
i. an outer member disposed co-axially with an inner member for insertion of said pessary into said vaginal cavity;
ii. said inner member being slidable within said outer member
wherein a portion of said pessary remains in said outer member when said inner member is fully engaged with said outer member to define a remaining portion of said pessary, said remaining portion of said pessary requiring a force of less than about 0.04 N/mm to be removed from said outer member.

24. The pessary applicator according to claim 23 wherein said remaining portion of said pessary requires a force of less than about 0.03 N/mm to be removed from said outer member.

25. The pessary applicator according to claim 23 wherein said remaining portion of said pessary requires a force of less than about 0.02 N/mm to be removed from said outer member.

26. The pessary applicator according to claim 23 wherein said exposed portion of said pessary requires a force of less than about 0.04 N/mm to be removed from said outer member.

27. The pessary applicator according to claim 23 wherein at least 10% of said pessary length remains in said outer member when said inner member is fully engaged with said outer member and said remaining portion of said pessary requires a force of less than about 0.04 N/mm to be removed from said outer member.

28. The pessary applicator according to claim 23 wherein at least 12% of said pessary length remains in said outer member when said inner member is fully engaged with said outer member and said remaining portion of said pessary requires a force of less than about 0.03 N/mm to be removed from said outer member.
